(19) United States
(12) Patent Application Publication
(10) Pub. No.: US 2005/0255163 A1
(43) Pub. Date: Nov. 17, 2005

(54) Co-grinding process for the preparation of a ternary composition

(76) Inventors: Fabio Carli, Trieste (IT); Paolo Corvi Mora, Piacenza (IT); Tiziana Canal, Trieste (IT)

Correspondence Address: Jospeh M Noto;Nixon Peabody, Clinton Square, PO Box 31051, Rochester, NY, 14603-1051, US

(21) Appl. No.: 10/515,097

(22) PCT Filed: May 19, 2003
(86) PCT No.: PCT/EP03/05241

§ 371 Date: Nov. 19, 2004

(30) Foreign Application Priority Data
May 20, 2002 (IT) MI02A001074

(51) Int. Cl. A61K 9/14; B02B 5/02

(52) U.S. Cl. 424/489; 241/22

(57) ABSTRACT

A ternary composition comprising an active substance, a hydrophilic or hydrophobic carrier and a co-grinding auxiliary substance and a process for the preparation thereof by the co-grinding of the thee components, in which said process allows operating with drastically reduced co-grinding times with respect to the known art and to obtain ternary compositions in which the active substance shows characteristics of amorphisation, solubility and dissolution speed as requested for the various uses.

### PRIOR ART

Processes of co-grinding of an active substance with a carrier are known in the scientific and patent literature.

The first suggestion relating to the use of the technique of co-grinding of a drug with a carrier, aimed at the improvement of the characteristics of passing into solution of poorly soluble drugs, dates from 1975 (Chem. Pharm. Bull., 23, 2973, 1975).

In this case a ball mill and linear hydrosoluble polyvinylpyrrolidone as a carrier were used.

The U.S. Pat. No. 4,639,370 describes co-grinding limited to mixtures of a poorly soluble drug with a carrier constituted of hydro-reswellable but insoluble cross-linked polymers, such as cross-linked polyvinylpyrrolidone, cross-linked sodium carboxymethylcellulose and cross-linked dextran.

Other materials suggested for use as carriers in the co-grinding technique have been cyclodextrines (DE 3427788) and silica gel (BP 1239893).

In these patents the process consists essentially in premixing the components and dry co-grinding them for a time necessary to obtain the desired degree of amorphisation and/or increase in dissolution speed. In many cases the necessary grinding time can be particularly long, reaching and sometimes exceeding 24 hours.

An improvement of the co-grinding process of a drug with a carrier has been introduced with the U.S. Pat. No. 5,354,560 and U.S. Pat. No. 5,449,521, wherein a process in which solvent vapour or water vapour is introduced into the co-grinding chamber of a high energy mill is described. The compositions obtained have an increased dissolution speed obtainable with reduced co-grinding times with respect to preceding patents.

Such a process is described further in the patent WO 963293, wherein products having improved characteristics are obtained by co-grinding using sodium starch glycolate as a carrier. It should be understood that, however, to obtain the desired result, the presence of water vapour in the co-grinding chamber is however necessary, vapour which is formed from the breaking off from the water adsorbed onto the carrier.

### SUMMARY OF THE INVENTION

A dry co-grinding process which allows the attainment of ternary compositions in very short times has now surprisingly been found in which to an active substance are conferred the characteristics of amorphisation, solubility and dissolution speed desired for various applications.

Said process is characterised in that the co-grinding mixture comprises, besides an active substance and a carrier, also a co-grinding auxiliary substance. Therefore the process according to the present invention consists in the dry grinding of a ternary mixture constituted by an active substance, a hydrophilic or hydrophobic carrier and a co-grinding auxiliary substance.

With this process the desired characteristics of the composition are obtained with very much shorter grinding times with respect to the times required for the binary compositions of the known art constituted by active substance and carrier.

### BRIEF DESCRIPTION OF THE FIGURES

FIG. 1 represents the enthalpy of fusion (J/g) for the ternary composition of example 1 (diagram (a)) and for the comparative binary composition of example A (diagram (b)).

FIG. 2 represents the enthalpy of fusion (J/g) for the ternary composition of example 2 (diagram (a)) and for the comparative binary composition of example B (diagram (b)).

FIG. 3 represents the enthalpy of fusion (J/g) for the ternary composition of example 3 (diagram (a)) and for the comparative binary composition of example C (diagram (b)).

FIG. 4 represents the percentage of residual crystallinity (% Cr) as a function of co-grinding time for the ternary composition of example 7 (curve (b)) and for the same ternary composition of examples 8 and 9 carried out at a higher grinding frequency (curves (c) and (d)), in comparison with the binary composition of example G (curve (a)), calculated on the basis of the differential scanning calorimetry data.

FIG. 5 represents the percentage of active substance (DHEA) released under sink conditions from the ternary composition of example 1 (curve (a)), in comparison with the percentage release of DHEA as it is (curve (b).

FIG. 6 represents the solubilisation kinetics (non sink conditions) of DHEA from the ternary composition of example 1 (curve (a) in comparison with DHEA as such (curve (b)).

FIG. 7 represents the percentage of active substance (progesterone) released under sink conditions from the composition of example 5 (curve (c)) in comparison with the percentage released from the composition of example E (curve (b) and from progesterone as such (curve (a)).

### DETAILED DESCRIPTION OF THE INVENTION

The characteristics and the advantages of the process according to the present invention and of the compositions obtained with said process, will be illustrated over the course of the following detailed description.

The process according to the present invention is carried out through the following treatment:
an active substance, a hydrophilic hydrophobic carrier and a co-grinding auxiliary substance all in powder form are dry co-ground in a mill. Optionally, such substances can be premixed in a conventional mixer.

At the end of the co-grinding process, the ternary composition obtained is unloaded from the mill and optionally sieved to eliminate possible aggregates.

The product of the present invention is a ternary composition in the form of finely subdivided powder, comprising an active substance, a hydrophilic or hydrophobic carrier and a co-grinding auxiliary substance, in which the active substance reduces its degree of crystallinity using considerably lower levels of grinding energy with respect to the prior art. Furthermore, in the case in which the active substance is constituted by a hydrophobic drug, a strong increase in solubility and/or solubilisation speed can be obtained using hydrophilic carriers whilst in the case in which the active substance is constituted by a hydrophilic drug a slowing down of the solubilisation speed can be obtained with respect to the active substance as such using hydrophobic carriers.

With the process according to the present invention the three components of the composition are closely absorbed one into the other at the nanocrystalline level. Furthermore the co-grinding auxiliary substance introduces an additional steric deformation factor into the crystalline matrix of the other components which adds to the mechanico-chemical activation thus facilitating the destructurisation and therefore allowing the use of lower energies or times of grinding.

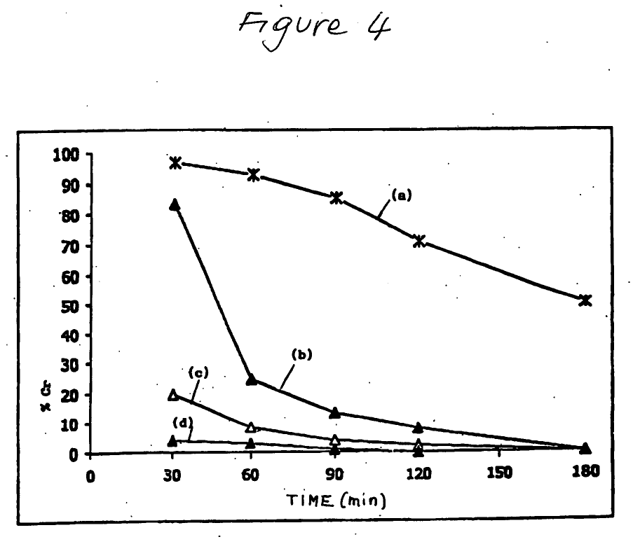

The presence of the co-grinding auxiliary substance brings about a drastic reduction of the energy necessary to transform the process from a simple grinding aimed at the reduction of the dimensions of the particles, to a mechanico-chemical process of amorphisation of the active substance. That is clearly demonstrated in FIG. 4 in which the residual crystallinity data are reported as a function of the co-grinding time of the DHEA/• cyclodextrine binary composition prepared according to the known art (curve (a)) and the corresponding DHEA/• cyclodexlrine/glycine ternary composition according to the present invention (curve (b)), in which the co-grinding auxiliary substance is constituted by glycine.

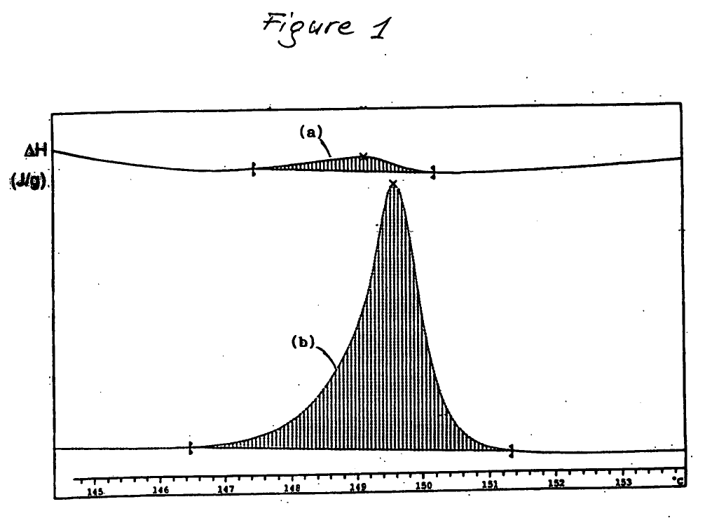

Both the mixtures have been subjected to co-grinding in a coaxial vibrational micromill at the lowest possible frequency, i.e. at a frequency of 10 Hz. The DHEA/β cyclodextrine/glycine ternary mixture has a completely different activation curve with respect to the DHEA/β cyclodextrine binary mixture. The addition of glycine to the DHEA/β cyclodextrine binary mixture makes it so that even at these low energy values the activation curve shows an exponentially falling progression of the residual crystallinity of the drug, a progression which is maintained even at higher frequencies. The ternary mixture has in fact been subjected to activation even at frequencies of 15 and 24 Hz which lead respectively to the curves (c) and (d). FIG. 4 clearly highlights the catalytic role of the co-grinding auxiliary substance in the mechanico-chemical inclusion process of the drug into the carrier, with the consequent improvement of the destructurisation process of the drug crystal. Such a role is highlighted also in the thermograms reported in FIGS. 1, 2 and 3 in which it is clear that, under equal operating conditions, the residual crystallinity and the fusion temperature of the active substance are clearly lower in the ternary composition with respect to the corresponding binary composition obtained according to the known art. The formation of the ternary composition according to the present invention produces in addition a clear cut change of the physico-chemical characteristics of the active substance with consequent modifications, for example, of the property of passage into solution. Preferably, in the case of an active substance poorly soluble in aqueous environments, a hydrophilic carrier is used with the aim of masking the hydrophobicity of the active substance through absorption inside the hydrophilic structure of the carrier with the consequent improvement in the solubility and dissolution speed.

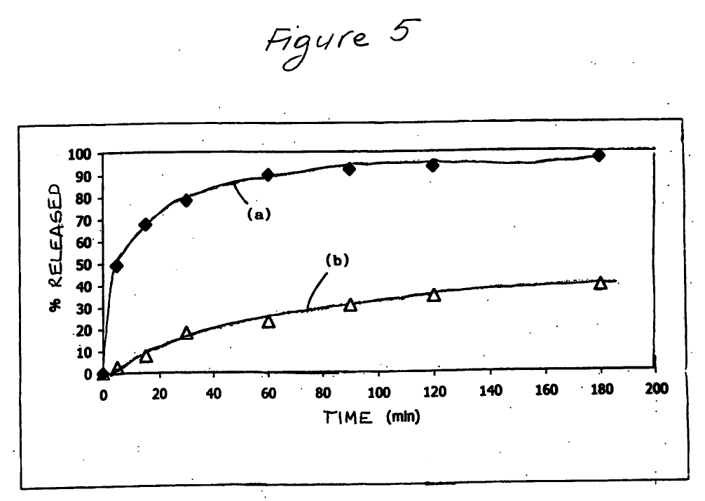

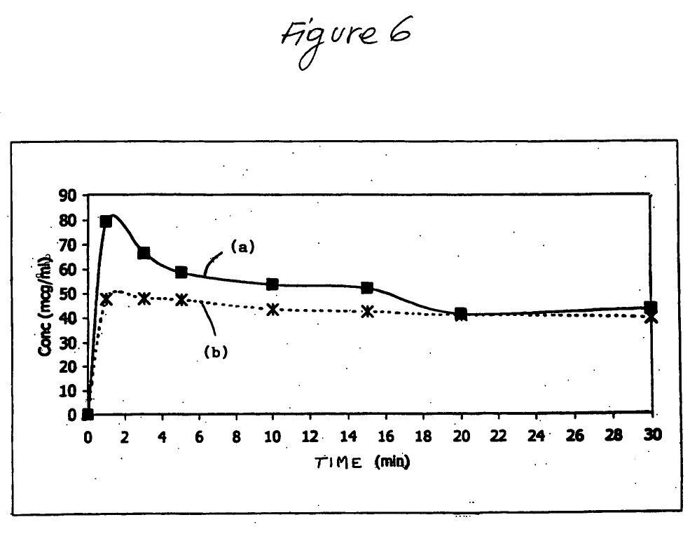

In FIGS. 5 and 6 are reported respectively the dissolution speed curves under sink conditions (% age of active substance released against time) and the to solubilisation kinetics curves under non sink conditions (concentration of active substance against time) of a hydrophobic drug (DHEA)/hydrophilic carrier combination. The dissolution speed of the DHEA from the ternary composition is markedly greater with respect to that of the DHEA as such, reaching 70% release after 15 minutes and 90% after 60 minutes, against the respective 9% and 20% for DHEA as such. Regarding the kinetics, in the ternary composition, the oversaturation reached by DHEA, which reaches a concentration of over 80 μg/ml in 2 minutes against around 50 μg/ml of DHEA as such, is evident.

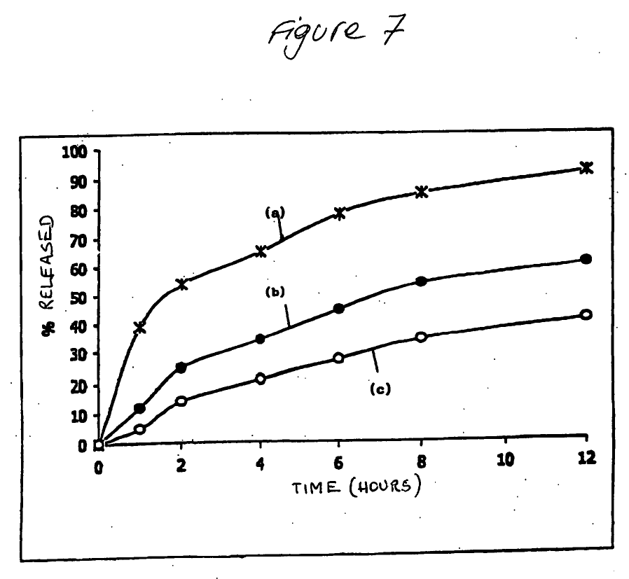

In the case instead, of an active substance very soluble in aqueous environments, a hydrophobic carrier is preferably used with the aim of masking the hydrophilicity of the active substance obtaining a slowing down of the dissolution speed in aqueous medium and therefore a controlled release composition with a duration of several hours can be obtained. In FIG. 7 is highlighted how the effect of the co-grinding auxiliary substance allows the hydrophobicity of the carrier to mask the hydrophilicity of the drug, with a consequent neat slowing down of the dissolution speed until the system assumes actual controlled release characteristics over a prolonged timespan of hours.

Amongst the hydrophilic, linear and cross-linked type carriers, cyclodextrines and cyclodextrine derivatives, dextrans, linear and cross-linked polyvinylpyrrolidone, cellulose and derivatives, polyacrylic acids, mannoglucuronans, chitosans, galactomannans and sodium starch glycolate can be mentioned by way of non exhaustive examples.

Non limiting examples of linear and cross-linked hydrophobic type carriers, are ethylcelluloses, polymethacrylates, polymethylmethacrylates, and polystyrene.

As co-grinding auxiliary substances, natural amino acids, and their derivatives; weak acids, such as for example malic acid, fumaric acid, ascorbic acid, citric acid; polyalcohols and derivatives; chelating agents, such as disodium ethylenediaminetetra-acetate; non-ionic, anionic or cationic surfactants, as well as lecithins, phospholipids and their semisynthetic or synthetic derivatives can be mentioned by way of non exhaustive examples.

The amino acids and disodium ethylenediamihetetra-acetate are the preferred co-grinding auxiliary substances. Glycine, lysine, serine and disodium ethylenediaminetetra acetate are particularly preferred.

Numerous active substances are suitable for to use in the compositions of the invention and comprise substances which act on the central nervous system and on the peripheral nervous system, cardiovascular system, hypotensives, diuretics, anti-inflammatory agents, analgesics, antipyretics, antiasthmatics, bronchodilators, anticough agents, mucolytics, antibiotics, chemotherapeutics, antivirals, hormones, antineoplastics, immunosuppressants, immunostimulants, photo-protectors and photoimmuno-protectors, peptides, polypeptides, proteins, vaccines etc.

Amongst the substances which can be used according to the invention, as non exhaustive examples can be mentioned:

Ergot alkaloids and derivatives, dihydroergotamine, dihydroergotoxin, bromocryptine and analogues and/or derivatives thereof.

Analgesics and non steroidal anti-inflammatory agents and salts thereof: sodium diclophenac, hydroxyethyl diclophenac pyrrolidine, diethylamine diclophenac, ibuprofen, flurbiprofen, ketoprofen, indomethacin, mefenamic acid, naproxene, nimesulide, piroxicam, celecoxib, valdecoxib and analogues and/or derivatives thereof.

Antiarythmics: amiodarone, di-isopyramide, propranolol, verapamil and analogues and/or derivatives thereof.

Antibacterials: Amoxycillin, flucloxacillin, gentamicin, rifampacin, erythromycin, cefalosporins and analogues and/or derivatives thereof.

Antifungals and antipsoriatics: amphotericin, butoconazole nitrate, ketoconazole, econazole, atretinate, fluconazole, flucitosine, griseofulvine, itraconazole, miconazole, nystatin, sulconazole, thioconazole and analogues and/or derivatives thereof.

Antivirals: acyclovir, gancyclovir, AZT, protease inhibitors and analogues and/or derivatives thereof.

Antihypertensives: amlodipine, clonidine, diltiazem, felodipine, guana-benzacetate, isradipine, minoxidil, nicardipine hydrochloride, nimodipine, nifedipine, prazosin hydrochloride, papaverine and analogues and/or derivatives thereof.

Antidepressants: carbamazepine and analogues and/or derivatives thereof.

Antihistamines: diphenidramine, chlorpheniramine, pyrilamine, chlorcyclizine, promethazine, acrivastine, cinnarizin, loratadine, terfenadine and analogues and/or derivatives thereof.

Antineoplastics and immunosuppressants: cyclosporin, dacarbazine, etretinate, etoposide, lomustine, melphalan, mitomycin, mitoxantrone, paclitaxel, procarbazine, tamoxifen, taxol and derivatives, taxotere and analogues and/or derivatives thereof.

Anxiolytics, sedatives, hypnotics: alprazolam, bromazepam, diazepam, lorazepam, oxazepam, temazepam, sulpiride, triazolam and analogues and/or derivatives thereof.

β-blockers: alprenolol, atenolol, oxprenolol, pindolol, propranolol and analogues and/or derivatives thereof.

β-agonists: salbutamol, salmeterol and analogues and/or derivatives thereof.

Cardiac inotropics and cardiovascular agents: amrinone, digitoxin, digoxin, lanatoside C, medigoxin, ubidecarenone and analogues and/or derivatives thereof.

Corticosteroids: beclomethasone, betamethasone, budesonide, cortisone acetate, desoxymethasone, dexamethasone, fludrocortidone acetate, flunisolide, hydrocortisone, methylprednisone, triamcinolone and analogues and/or derivatives thereof.

Gastrointestinal and anti H₂-histamines: cimetidtne, cisapride, domperidone, famotidine, loperamide, mesalazine, omeprazol, ondansetron hydrochloride, ranitidine and analogues and/or derivatives thereof.

Hypolipidemics: bezafibrate, clofibrate, gemfibrozil, probucol, lovastatin and analogues and/or derivatives thereof.

Anti-angina agents: amyl nitrate, glyceryl trinitrate, isosorbide dinitrate and mononitrate, pentaerythritol tetranitrate and analogues and/or derivatives thereof.

Centrally acting drugs: e.g. nicotine.

Vitamins and nutritional agents: beta-carotene, vitamin A, vitamin B₂, vitamin D and derivatives, vitamin E and derivatives, vitamin K and analogues and/or derivatives thereof.

Opioid analgesics: codeine, dextropropoxyphene, dihydrocodeine, morphine, pentazocine, methadone and analogues and/or derivatives thereof.

Sexual hormones: danazol, ethinylestradiol, medroxyprogesterone acetate, methyltestosterone, noretisterone, norgestrel, estradiol, estriol, progesterone, stilbestrol, diethylstilbestrol and analogues and/or derivatives thereof.

Peptide, proteic or polysaccharidic molecules with various activities: leuprolide and LH-RH analogues, calcitonin, glutathione, somatotropin (GH), somatostatin, desmopressin (DDAVP), interferon, molgramostim, epidermal growth factor. (EGF), nerve growth factor (NGF), insulin, glucagons, toxins or toxoids (e.g. tetanus toxin), proteic or polysaccharidic type antigenic factors, low molecular weight heparin, heparinoids and analogues and/or derivatives thereof.

Furthermore, any cosmetic active substance can be used, such as for example agents for solar protection, anti ageing agents, coadjuvant agents for the treatment of dermatological diseases.

Finally, any active substance suitable for dietary integration can be used.

The weight ratio of active substance and carrier is comprised of between 1:0.1 and 1:100 and preferably between 1:0.5 and 1:50.

The weight ratio of the active substance and the co-grinding auxiliary substance is comprised of between 1:0.1 and 1:20 and preferably between 1:0.2 and 1:10.

For the co-grinding process. according to the present invention, equipment normally employed for the grinding of powders are used, such as for example ball mills, blade mills, vibrational mills, centrifugal mills and planetary mills. Preferably, mills capable of reaching high energies are used. Low energy mills can nevertheless be also used. In fact, the catalysing action of the co-grinding auxiliary substance allows, with the appropriate selection of the three substances to be treated, the use of mills with even lower energy with respect to these used in the preceding art. Generally, it is preferred to use equipment in which it is possible to expose the powder mixture to the mechanical shock action generated by the specific mill used and by the means of grinding. Regarding the optional steps of the process, which are-for example the premixing of the powders and sieving, equipments known in the art can be used, which are for example these described in the text “The theory and practice of industrial pharmacy”, L. Lachman and H. A. Lieberman, 1986.

The co-grinding time depends on the characteristics of the component substances, the energy applied and the degree of amorphisation desired and in general is comprised of between 0.25 and 24 hours.

Preferably the co-grinding time is no greater than 10 hours.

The compositions obtained by operating according to the examples reported below have been characterised by DSC and from the enthalpy of fusion the percentage residual crystallinity has been calculated.

Furthermore, the percentage of active substance released under sink conditions and the solubilisation kinetics under non sink conditions have been determined.

With the process of the present invention, one obtains ternary compositions comprising an active substance, a hydrophilic or hydrophobic carrier and a co-grinding auxiliary substance in which the characteristics, which are solubility, dissolution speed, solubilisation kinetics can be modified in different measures according to the requirements for usage. For example, a high dissolution speed for when a rapid action of the active substance is required, or a low dissolution speed for when a delayed action is required can be obtained.

A further advantage with respect to the prior art is given by the increased versatility of the process. In fact, contrarily to the prior art, the catalysing effect of the co-grinding auxiliary substance is effective even with the hydrophilic drug/hydrophobic carrier combination.

A further advantage of the present invention is given by the fact that, being able to use lower energy levels and/or lower grinding times, even substances poorly stable from the physico-chemical point of view, for example thermolabile substances, can be activated. Furthermore, the considerably reduced grinding times result in an improvement in the efficiency of the plants.

Finally, the present invention even allows the use of mills capable of developing lower energies than these used in the prior art.

The compositions obtained with the process according to the present invention, in powder form, can be packaged into sachets as such or in mixtures with pharmaceutically acceptable excipients. In addition, they can be incorporated into other formulations such as creams, ointments, pastes, gels, suppositories, ovules, transdermal plasters, etc. Moreover, they can be formulated with excipients and incorporated into cosmetic use formulations. Finally, they can be formulated for alimentary integration in mixtures with excipients, acceptable from the alimentary point of view. With the aim of non exhaustive illustration of the present invention, the following experimental examples are reported.

### EXAMPLE 1

1 kg of DHEA(dehydroepiandrosterone)/α cyclodextrine/glycine mixture in the ratio 1/2/3 M is homogenised for 10 minutes in a rotating body mixer for powders. The mixture is loaded into a vibrational mill equipped with sintered alumina cylindrical means of grinding and subjected to grinding with a vibrational amplitude comprised of between 6 and 10 mm for 2 hours.

The product obtained, with a yield of 98.8%, is sieved through a 355 micrometer sieve and 99.9% of the product is recovered in the form of a finely subdivided flowing powder.

The product obtained has a DHEA/α cyclodextrine/glycine composition with a molar ratio of 1/2/3.

The results of the characterisation of said product are reported in table 1, in FIG. 1 (curve (a)) and in FIGS. 5 and 6 (curve (a)).

### EXAMPLE 2

2.46 g of xibornol, 10.80 g of β-cyclodextrine and 1.74 g of lysine hydrochloride are loaded into a jar of a planetary mill containing 16 agate balls and subjected to grinding, at room temperature, at a rotational velocity of 150 rpm for 1 hour.

The product obtained is sieved and 99.4% of product is recovered in the form of finely subdivided flowing powder.

The product obtained has a xibornol/β-cyclodextrine/lysine molar ratio of 1/1/1.

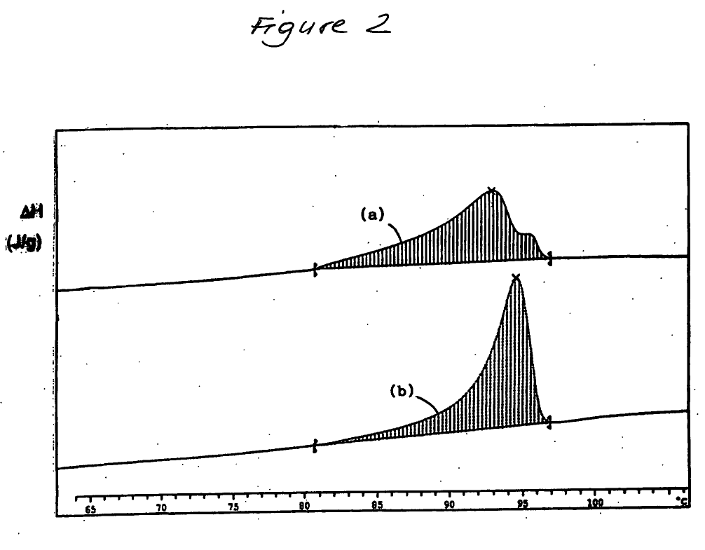

The results of the characterisation are reported in table 1 and in FIG. 2 (curve (a)).

### EXAMPLE 3

15 g of a 1/1/2 M mixture of buytl-methoxydibenzoyl methane, β-cyclodextrine and serine are treated according to the operative method of example 2 the only difference being that the grinding is carried out for 2 hours at 200 rpm.

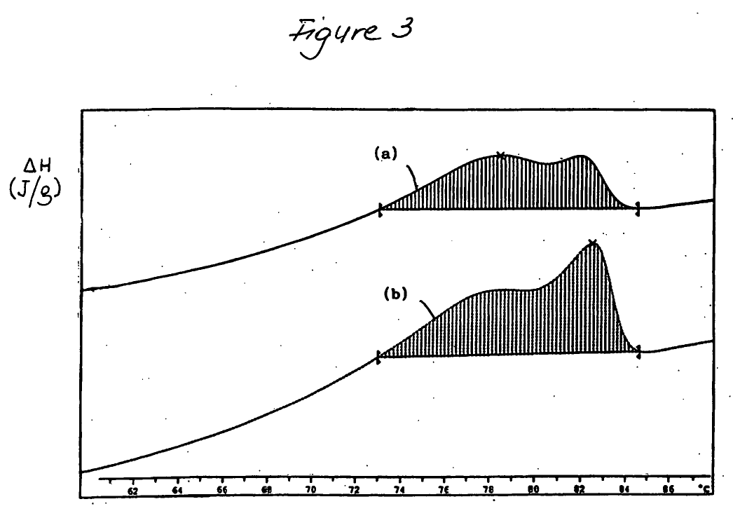

The results of the characterisation of the product obtained are reported in table 1 and in FIG. 3 (curve (a)).

### EXAMPLE 4

A binary mixture constituted of hydrocortisone acetate and cross-linked polyvinylpyrrolidone in the weight ratio of 1/3 is added to with glycine in such a quantity as to obtain a molar ratio of 2/1 with the hydrocortisone acetate. 1 gram of the mixture obtained is loaded into a 25 ml stainless steel jar of a coaxial vibrational micromill and subjected to grinding, with a vibration frequency of 15 Hz for 1 hour.

The product obtained is sieved and is recovered in a quantity of 98.3% in the form of a finely subdivided flowing powder.

The results of the characterisation of the composition obtained are reported in table 1.

### EXAMPLE 5

A 1 g sample of progesterone/polymethylmethacrylate binary mixture in a weight ratio 1/1.5 is added to with glycine in such a quantity as to obtain a 1/3 M ratio with the progesterone. 1 g of the mixture obtained is subjected to grinding in a coaxial vibrational micromill with a frequency of 20 Hz for 1 hour. The ternary product thus obtained is sieved at 355 μm and is recovered in a quantity of 98.7% in the form of a finely subdivided flowing powder.

The percentage progesterone release from the composition obtained is reported in FIG. 7, curve (c).

In the same figure, the percentage release of progesterone from the binary composition of example E (curve (b)) and of progesterone as it is (curve (a)) are also reported.

The composition of example 5 has, therefore, the sense of a delayed release composition.

### EXAMPLE 6

A resveratrol/linear polyvinylpyrrolidone binary mixture in the weight ratio of 1/3 is added to with serine in such a quantity as to obtain a 1/2 M ratio with resveratrol. 15 g of the mixture obtained are loaded into a jar of a planetary mill and subjected to grinding for 2 hours at 100 rpm. At the end of the process, the product obtained is sieved through a 355 μm sieve and 99.1% of the product is recovered in the form of a finely subdivided flowing powder.

The results of the characterisation of the product are reported in table 1.

### EXAMPLE 7

A 1 g sample of DHEA/β-cyclodextrine/glycine ternary mixture in the ratio 1/1/2 M is subjected to grinding in a coaxial vibrational micromill with a frequency of 10 Hz for three hours. Samples are removed at 30, 60, 90, 120 and 180 minutes for the determination of residual crystallinity. The final product is sieved and 98.8% of the product is recovered in the form of a finely subdivided flowing powder.

### EXAMPLE 8

Example 7 is repeated with the difference being that the frequency is 15 Hz.

### EXAMPLE 9

Example 7 is repeated with the difference that the frequency is 24 Hz. The percentages of DHEA residual crystallinity in the compositions obtained as a function of the grinding times of examples 7, 8 and 9 are reported in FIG. 4, respectively in the curves (b), (c) and (d), in comparison with the binary composition of example G (curve (a)).

### EXAMPLE 10

1 kg of N-acetyl cystein and disodium ethylenediamine tetra acetate (NaEDTA) mixture in the ratio 8/1 M is treated in a vibrational mill equipped with sintered alumina cylindrical means of grinding with a vibrational amplitude comprised of between 6 and 8 mm for 30 minutes. At the end of the treatment is added α-cyclodextrine in such a quantity as to obtain a ternary mixture in which the N-acetyl cystein/NaEDTA/α-cyclodextrine ratio is 50.0/15.6/34.4% w/w and is co-ground for a further 30 minutes.

The product obtained, with a yield of 99.5%, is sieved through a 355 micrometer sieve and 99.8% of the product is recovered in the form of a finely subdivided flowing powder.

The results of the characterisation of the composition obtained are reported in table 1.

### EXAMPLE 11

1 kg of a mixture of 2-phenyl benzamidazol-5-sulphonic acid, β-cyclodextrine and L-arginine in the weight ratio 30/50/20 is homogenised for 10 minutes in a rotating body mixer for powders. The mixture is loaded into a vibrational mill equipped with sintered alumina cylindrical means of grinding and subjected to grinding with a vibrational amplitude comprised of between 6 and 10 mm for 1 hour.

The product obtained, with a yield of 97.8%, is sieved and 99.6% of product is recovered in the form of a finely subdivided flowing powder.

The solubility in water at 50° C. has resulted being approx. 80 mg/ml.

### EXAMPLES A-I

With the aim of comparison with the examples 1-7, 10 and 11 relating to (active ingredient/carrier/co-grinding auxiliary substance) ternary mixtures, examples A-I relating to the corresponding (active ingredient/carrier) binary mixtures have been carried out.

### EXAMPLE A

129 g of DHEA and 871 g of α-cyclodextrine (ratio 1/2 M) are treated as in example 1.

The results of the characterisation of the composition obtained are reported in table 1 and in FIG. 1 (curve (b)).

### EXAMPLE B

2.78 g of xibornol and 12.22 g of β-cyclodextrine (ratio 1/1 M) are treated as in example 2.

The results of the characterisation are reported in table 1 and in FIG. 2 (curve (b)).

### EXAMPLE C

15 g of a 1/1 M mixture of butyl-methoxydibenzoyl methane and β-cyclodextrine are treated as in example 3.

The results of the characterisation of the composition obtained are reported in table 1 and in FIG. 3 (curve (b)).

### EXAMPLE D

1 g of a mixture constituted by hydrocortisone acetate and cross-linked polyvinylpyrrolidone in the weight ratio 1/3 is treated as in example 4.

The results of the characterisation of the composition obtained are reported in table 1.

### EXAMPLE E

A 1 g sample of progesterone/polymethylmethacrylate binary mixture in the weight ratio 1/1.5 is treated as in example 5. The percentage release of progesterone from the composition obtained is represented in FIG. 7, curve (b).

### EXAMPLE F

A 15 g sample of resveratrol/linear polyvinylpyrrolidone binary mixture in the weight ratio 1/3 is treated as in example 6. The results of the characterisation of the composition obtained are reported in table 1.

### EXAMPLE G

A 1 g sample of DHEA/β-cyclodextrine binary mixture in the ratio 1/1 M is treated as in example 7. The percentage of residual crystallinity of DHEA in the composition obtained, as a function of the grinding time, is reported in FIG. 4, curve (a).

### EXAMPLE H

A sample of N-acetyl cystein/α-cyclodextrine binary mixture in the weight ratio 1:1.45 is treated as in example 10.

The results of the characterisation of the composition obtained are reported in table 1.

### EXAMPLE I

A sample of the binary mixture constituted by 2-phenyl benzamidazol-5-sulphonic acid and β-cyclodextrine in the weight ratio 30/50 is treated as in example 11.

The water solubility of the product obtained was less than 1 mg/ml.

TABLE 1

| | | | | Residual | |
|---|---|---|---|---|---|
| | | | Active | Enthalpy | |
| | | | Substance | (J/g) | % age |
| | | | Enthalpy | Following | Residual |
| Composition | FIGS. | Example | (J/g) | Treatment | Crystallinity |
| (DHEA*/α-cyclodextrine/glycine) | 1 (a) | Ex 1 | 100 | 0.6 | 0.6 |
| (DHEA/α-cyclodextrine) | 1 (b) | Ex A | | 9.6 | 9.6 |
| (Xibornol/β-cyclodextrine/lysine) | 2 (a) | Ex 2 | 92.8 | 42.5 | 45.8 |
| (Xibornol/β-cyclodextrine) | 2 (b) | Ex B | | 52.3 | 56.4 |
| BMDM**/β-cyclodextrine/serine) | 3 (a) | Ex 3 | 62.4 | 27.8 | 44.6 |
| BMDM**/β-cyclodextrine) | 3 (b) | Ex C | | 43.8 | 70.2 |
| HCA***/cIPVP****/glycine) | / | Ex 4 | 117.9 | 0.0 | 0.0 |
| HCA***/cIPVP****) | / | Ex D | | 64.4 | 54.5 |
| (Resveratrol/PVP K30\hat{ }/serine) | / | Ex 6 | 317.7 | 6.4 | 2.0 |
| (Resveratrol/PVP K30) | / | Ex F | | 11.6 | 3.6 |
| (NAC\hat{ }\hat{ }/NaEDTA\hat{ }\hat{ }\hat{ }/α-cyclodextrine) | / | Ex 10 | 192.9 | 122.4 | 63.4 |
| (NAC/α-cyclodextrine) | / | Ex H | | 174.5 | 90.5 |

*dehydroepiandrosterone
\hat{ }polyvinylpyrrolidone
**butylmethoxy dibenzoylmethane
\hat{ }\hat{ }N-acetyl cystein
***hydrocortisone acetate
\hat{ }\hat{ }\hat{ }disodium ethylenediamine tetra acetate

1. A process of dry co-grinding of a ternary mixture consisting in an active substance, a hydrophilic or hydrophobic carrier, characterised in that said mixture comprises as third component a co-grinding auxiliary substance suitable for reducing the co-grinding time selected from the group comprising aminoacids, malic acid, fumaric acid, ascorbic acid, citric acid, polyalcohols, disodium ethylenediamine tetra-acetate, surfactants, lecithins, phospholipids and derivatives thereof.

2. The process according to claim 1, wherein said co-grinding time is comprised between 0.25 and 10 hours.

3. The process according to claim 1, wherein said hydrophilic carrier is selected from the group comprising cyclodextrines, derivatives of cyclodextrines, dextrans, linear and cross-linked polyvinylpyrrolidone, cellulose and cellulose derivatives, manno glucuronans, chitosans, galactomannans and sodium starch glycolate.

4. The process according to claim 1, wherein said hydrophobic carrier is selected from the group comprising ethylcellulose, polymethacrylates, polymethylmethacrylates and polystyrene.

5. (canceled)

6. The process according to claim 1, wherein said co-grinding auxiliary substance is selected from the group comprising glycine, lysine, serine and disodium ethylenediamine tetra-acetate.

7. The process according to claim 1, wherein the weight ratio of said active substance and said carrier is comprised between 1:0.1 and 1:100.

8. The process according to claim 1, wherein the weight ratio of said active substance and said carrier is comprised between 1:0.5 and 1:50.

9. The process according to claim 1, wherein the weight ratio of said active substance and said co-grinding auxiliary substance is comprised between 1:0.1 and 1:20.

10. The process according to claim 1, wherein the weight ratio of said active substance and said co-grinding auxiliary substance is comprised between 1:0.2 and 1:10.

11. A ternary composition in the form of finely subdivided flowing powder obtainable by the process according to claim 1, consisting in an active substance, a hydrophilic or hydrophobic carrier and as third component a co-grinding auxiliary substance suitable for reducing the co-grinding time selected from the group comprising aminoacids, malic acid, fumaric acid, ascorbic acid, citric acid, polyalcohols, disodium ethylenediamine tetra-acetate, surfactants, lecithins, phospholipids and derivatives thereof, characterised in that said ternary composition has an enthalpy and a residual crystallinity lower than the corresponding binary composition free of the co-grinding auxiliary substance.

12. The composition according to claim 11, wherein said hydrophilic carrier is selected from the group comprising cyclodextrines, derivatives of cyclodextrines, dextrans, linear and cross-linked polyvinylpyrrolidone, cellulose and derivatives of cellulose, mannoglucuronans, chitosans, galactomannans and sodium starch glycolate.

13. The composition according to claim 11, wherein said hydrophobic carrier is selected from the group comprising ethylcellulose, polymethacrylates, polymethylmethacrylates and polystyrene.

14. (canceled)

15. The composition according to claim 11, wherein said active substance is selected from the group comprising drugs which act on the central nervous system and on the peripheral nervous system, cardiovascular drugs, hypotensives, diuretics, anti-inflammatory agents, analgesics, antipyretics, antiasthmatics, bronchodilators, anticough agents, mucolytics, antibiotics, chemotherapeutics, antivirals, hormones, antineoplastics, immunosuppressants, immunostimulants, photo protectors and immunophoto protectors, peptides, polypeptides, proteins and vaccines.

16. The composition according to claim 11, wherein said active substance is selected from the group of the cosmetic active substances comprising agents for solar protection, anti ageing agents and co-adjuvant agents for the treatment of dermatological diseases.

17. The composition according to claim 11, wherein said active substance is selected from the group of the active substances suitable for alimentary integration.

18. The composition according to claim 11, wherein said active substance is a substance poorly soluble in aqueous environments and said carrier is a hydrophilic carrier.

19. The composition according to claim 11, wherein said active substance is a substance soluble in aqueous environments and said carrier is a hydrophobic carrier.

20. The composition according to claim 11, wherein the weight ratio of said active substance and said carrier is comprised between 1:0.1 and 1:100.

21. The composition according to claim 11, wherein the weight ratio of said active substance and said carrier is comprised between 1:0.5 and 1:50.

22. The composition according to claim 11, wherein the weight ratio of said active substance and said co-grinding auxiliary substance is comprised between 1:0.1 and 1:20.

23. The composition according to claim 11, wherein the weight ratio of said active substance and said co-grinding auxiliary substance is comprised between 1:0.2 and 1:10.

24. The composition according to claim 11, wherein said composition is packaged as is in sachets or in capsules.

25. The composition according to claim 11, wherein said composition is transformed into finished pharmaceutical forms in mixtures with pharmaceutically acceptable excipients.

26. The composition according to claim 11, wherein said composition is transformed into finished cosmetic forms in mixtures with cosmetically acceptable excipients.

27. The composition according to claim 11, wherein said composition is transformed into forms suitable for to alimentary integration in mixture with acceptable excipients, from the alimentary point of view.

28. The process according to claim 1, wherein said co-grinding time is comprised between 0.25 and 24 hours.
